(19) United States
(12) Patent Application Publication
(10) Pub. No.: US 2025/0069730 A1
(43) Pub. Date: Feb. 27, 2025

(54) MEDICAL IMAGE PROCESSING DEVICE, TREATMENT SYSTEM, MEDICAL IMAGE PROCESSING METHOD, AND STORAGE MEDIUM

(71) Applicants: TOSHIBA ENERGY SYSTEMS & SOLUTIONS CORPORATION, Kawasaki-shi (JP); National Institutes for Quantum Science and Technology, Chiba-shi (JP)

(72) Inventors: Ryusuke HIRAI, Meguro Tokyo (JP); Keiko OKAYA, Setagaya Tokyo (JP); Shinichiro MORI, Chiba-shi (JP)

(73) Assignees: TOSHIBA ENERGY SYSTEMS & SOLUTIONS CORPORATION, Kawasaki-shi (JP); National Institutes for Quantum Science and Technology, Chiba-shi (JP)

(21) Appl. No.: 18/948,945

(22) Filed: Nov. 15, 2024

(30) Foreign Application Priority Data
May 19, 2022 (JP) 2022-082237

(51) Int. Cl. G16H 30/40

(52) U.S. Cl. CPC G16H 30/40

(57) ABSTRACT

A medical image processing device includes a first image acquirer, a second image acquirer, an area acquirer, an image similarity calculator, a cost calculator, and a registrator. The first image acquirer acquires a first image which is captured by imaging an internal body of a patient. The second image acquirer acquires a second image which is captured by imaging the internal body of the patient at a time point different from that of the first image. The area acquirer acquires two or more areas corresponding to the first image or the second image. The image similarity calculator calculates a similarity between the first image and the second image. The cost calculator calculates a cost based on a positional relationship between the areas. The registrator calculates a relative position of the first image with respect to the second image to increase the similarity and to decrease the cost.

### CROSS-REFERENCE TO RELATED APPLICATION

The present application claims priority based on Japanese Patent Application No. 2022-082237 filed May 19, 2022 and PCT/JP2023/004959 filed Feb. 14, 2023, the contents of which are incorporated herein by reference.

### FIELD

An embodiment of the present invention relates to a medical image processing device, a treatment system, a medical image processing method, and a storage medium.

### BACKGROUND

Radiation treatment is a treatment method of destroying a tumor (a lesion) in a patient's body by irradiating the tumor with radiation. Since radiation may affect normal tissue in a patient's body when the normal tissue is irradiated with the radiation, it is necessary to accurately irradiate a position of a tumor with radiation in the radiation treatment. Accordingly, when the radiation treatment is performed, for example, computed tomography (CT) is first performed in advance in a treatment planning stage, and a position of a tumor in the patient's body is three-dimensionally confirmed. An irradiation direction of radiation or an irradiation intensity of the radiation is planned on the basis of the confirmed position of a tumor. Thereafter, in a treatment stage, the position of the patient is aligned with the position of the patient in the treatment planning stage, and the tumor is irradiated with radiation according to the irradiation direction and the irradiation intensity planned in the treatment planning stage.

In alignment of a patient in the treatment stage, a misalignment in position of a patient between a transparent image of an internal body of the patient captured in a state in which the patient lies on a bed immediately before treatment starts and a digitally reconstructed radiograph (DRR) image obtained by virtually reconstructing a transparent image from a three-dimensional CT image captured at the time of treatment planning is calculated by comparing the images. By moving the bed on the basis of the calculated misalignment, positions of tumors, bones, and the like in the body of the patient are aligned with those at the time of treatment planning.

A misalignment in position of a patient is calculated by searching for positions in a CT image such that a DRR image most similar to a transparent image is reconstructed. In the related art, a plurality of methods of automating search for a position of a patient using a computer have been proposed. However, in the related art, the automatic search results are confirmed by causing a user (such as a doctor) to visually compare the transparent image with the DRR image.

At this time, it may be difficult to visually ascertain a position of a tumor appearing in the transparent image. This is because a tumor has higher radiolucency than bone or the like and thus a tumor does not appear clearly in the transparent image. Therefore, at the time of treatment, capturing a CT image instead of a transparent image to ascertain a position of a tumor is also performed. In this case, a misalignment in position of a patient is calculated by comparing a CT image captured at the time of treatment planning with a CT image captured in the treatment stage, that is, by comparing the CT images.

In comparison between CT images, a position most similar to that of one CT image is calculated while changing the position of another CT Image. An example of the method of comparing CT images is disclosed, for example, in Patent Document 1 (Japanese Patent No. 5693388). In the method disclosed in Patent Document 1, a position of an image which is most similar is searched for as a position of a tumor by preparing an image in the vicinity of a tumor included in a CT image captured at the time of treatment planning as a template and performing template matching of a CT image captured in the treatment stage. Then, a misalignment in position of the patient is calculated on the basis of the found position, and the position of the patient is aligned to have the same posture as that at the time of treatment planning by moving the bed according to the misalignment in the same way as described above. In the method disclosed in Patent Document 1, a search method of scanning the prepared template while changing the posture such as tilting the template as well as three-dimensionally scanning the prepared template is also mentioned.

However, in the method disclosed in Patent Document 1, it is considered to be important that a position in the vicinity of a tumor of interest is aligned with the CT image in the vicinity of the tumor prepared as a template. Accordingly, in the method disclosed in Patent Document 1, it cannot be said that positions of internal tissue of the patient other than the vicinity of the tumor are accurately aligned. That is, when a position of a patient is aligned using the method disclosed in Patent Document 1 and emitted radiation reaches the tumor, planned energy of the radiation may not be applied to the tumor according to the internal tissue of the patient in a route along which the radiation passes.

The radiation used for radiation treatment loses energy at the time of passing through matter. Accordingly, in the treatment planning in the related art, a radiation irradiation method is determined by virtually calculating an amount of energy loss of radiation to be emitted on the basis of a captured CT image. In consideration of this, alignment of internal tissue of the patient located in a route along which the radiation passes is also important at the time of alignment of the position of the patient in the treatment stage.

An example of a method of comparing CT images in consideration of this is disclosed, for example, in Patent Document 2 (United States Patent Application, Publication No. 2011/0058750). In the method disclosed in Patent Document 2, comparison between CT images is performed using CT images having been converted by calculating the arrival energy of radiation at each pixel. However, in the method disclosed in Patent Document 2, image comparison using a DRR image reconstructed from the converted CT image is performed at the time of performing image comparison. That is, in the method disclosed in Patent Document 2, the image used for image comparison has lost information of a three-dimensional image of the CT image.

A method of combining the method disclosed in Patent Document 1 with the method disclosed in Patent Document 2 and aligning a position of a patient through template matching using a converted CT image is conceivable. However, since the method of calculating the arrival energy changes according to an irradiation direction of radiation, it is necessary to recalculate the arrival energy whenever a posture of a template used for template matching is changed. Accordingly, even when the method disclosed in Patent Document 1 is combined with the method disclosed in Patent Document 2, it is necessary to prepare a plurality of templates according to postures or to align the position with a focus on the vicinity of a tumor, and thus it is not possible to easily perform alignment with internal tissue of a patient located in a route along which radiation passes.

Patent Document 3 (Japanese Unexamined Patent Application, First Publication No. 2022-029277) discloses a method of calculating a water-equivalent thickness associated with an amount of energy attenuation in a route along which radiation passes from a CT image and correcting a misalignment in position of a patient such that an amount of energy applied from radiation to a tumor approaches an amount of energy at the time of treatment planning.

In this way, in the alignment methods between CT images disclosed in Patent Documents 1 to 3, the alignment of CT images is not performed using positions of a tumor, an irradiation field, and a site called an organ at risk at the time of treatment in consideration of only a degree of coincidence between images. As a result, alignment accuracy between CT images may be low.

### BRIEF DESCRIPTION OF THE DRAWINGS

FIG. 1 A block diagram schematically showing a configuration of a treatment system including a medical image processing device according to a first embodiment.

FIG. 2 A diagram showing a CTV and a PTV in a treatment planning stage and a treatment stage.

FIG. 3 A block diagram schematically showing a configuration of the medical image processing device 100 according to the first embodiment.

FIG. 4 A diagram showing an example of a relationship between emission of radiation and an irradiation object of the radiation in the treatment system.

FIG. 5 A diagram showing another example of a relationship between emission of radiation and an irradiation target of the radiation in the treatment system.

FIG. 6 A flowchart showing an example of a process flow that is performed by the medical image processing device according to the first embodiment.

FIG. 7 A flowchart showing another example of a process flow that is performed by the medical image processing device according to the first embodiment.

FIG. 8 A block diagram schematically showing a configuration of a medical image processing device 100B according to a second embodiment.

FIG. 9 A flowchart showing a process flow that is performed by the medical image processing device 100B according to the second embodiment.

### DETAILED DESCRIPTION

Hereinafter, a medical image processing device, a treatment system, a medical image processing method, and a storage medium according to an embodiment will be described with reference to the drawings.

A medical image processing device according to an embodiment includes a first image acquirer, a second image acquirer, an area acquirer, an image similarity calculator, a cost calculator, and a registrator. The first image acquirer acquires a first image which is captured by imaging an internal body of a patient. The second image acquirer acquires a second image which is captured by imaging the internal body of the patient at a time point different from that of the first image. The area acquirer acquires two or more areas corresponding to one or both of the first image and the second image. The image similarity calculator calculates a similarity between the first image and the second image. The cost calculator calculates a cost based on a positional relationship between the areas. The registrator calculates a relative position of the first image with respect to the second image to increase the similarity and to decrease the cost.

### First Embodiment

### (Entire Configuration)

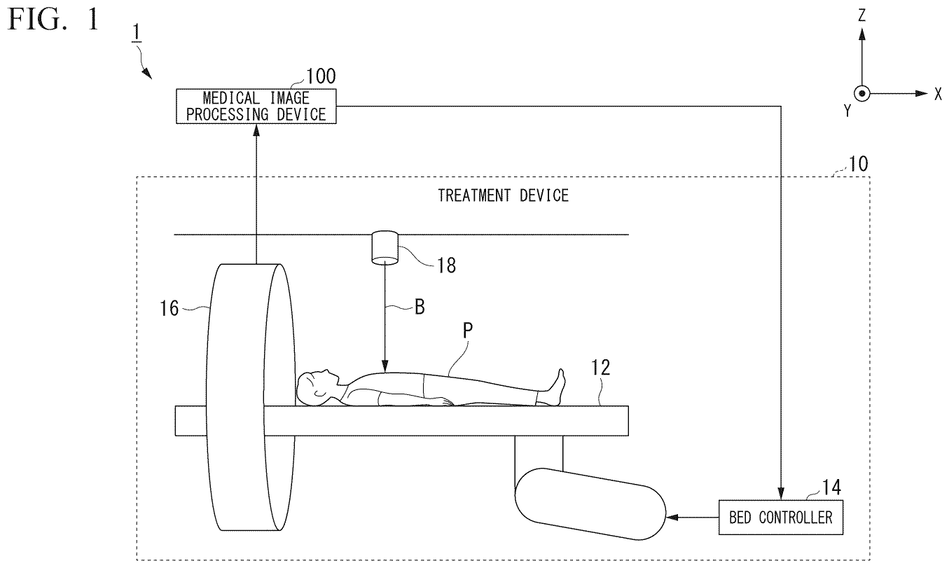

FIG. 1 is a block diagram schematically showing a configuration of a treatment system including a medical image processing device according to a first embodiment. The treatment system 1 includes, for example, a treatment device 10 and a medical image processing device 100. The treatment device 10 includes, for example, a bed 12, a bed controller 14, a computed tomography (CT) device 16 (hereinafter referred to as a “CT imaging device 16”), and a treatment beam emission gate 18.

The bed 12 is a movable treatment table on which a test subject (patient) P who is to be subjected to radiation treatment is fixed, for example, in a state in which the test subject is lying down using a fixture or the like. The bed 12 moves in a state in which a patient P is fixed in the CT imaging device 16 of a ring shape including an opening under the control of the bed controller 14. The bed controller 14 controls a translation mechanism and a rotation mechanism which are provided in the bed 12 such that a direction in which the patient P fixed to the bed 12 is irradiated with a treatment beam B is changed according to a movement amount signal output from the medical image processing device 100. The translation mechanism can drive the bed 12 in three axial directions, and the rotation mechanism can drive the bed 12 around three axes. Accordingly, the bed controller 14 controls the translation mechanism and the rotation mechanism of the bed 12, for example, such that the bed 12 moves in six decrees of freedom. The number of degrees of freedom in which the bed controller 14 controls the bed 12 may not be six and may be equal to or less than six (for example, four) or may be equal to or greater than six (for example, eight).

The CT imaging device 16 is an imaging device for performing three-dimensional computed tomography. In the CT imaging device 16, a plurality of radiation sources are provided inside of a ring-shaped opening, and radiation for seeing the inside of the body of the patient P is emitted from each radiation source. That is, the CT imaging device 16 emits radiations from a plurality of positions around the patient P. The radiation emitted from each radiation source in the CT imaging device 16 is, for example, an X-ray. The CT imaging device 16 detects radiation emitted from corresponding radiation sources, passing through the body of the patient P, and reaching a plurality of radiation detectors provided inside of the ring-shaped opening using the plurality of radiation detectors. The CT imaging device 16 generates a CT image of the internal body of the patient P on the basis of a magnitude of energy of radiation detected by each radiation detector. The CT image of the patient P generated by the CT imaging device 16 is a three-dimensional digital image in which the magnitude of energy of radiation is represented by a digital value. The CT imaging device 16 outputs the generated CT image to the medical image processing device 100. Three-dimensional imaging of the internal body of the patient P in the CT imaging device 16, that is, emission of radiation from each radiation source or generation of a CT image based on radiation detected by each radiation detector, is controlled, for example, by an imaging controller (not shown).

The treatment beam emission gate 18 emits radiation for destroying a tumor (a lesion) which is a treatment target site in the body of the patient P as a treatment beam B. Examples of the treatment B include an X-ray, a y-ray, an electron beam, a proton beam, a neutron beam, and a baryon beam. The patient P (more specifically, the tumor in the body of the patient P) is linearly irradiated with the treatment beam B from the treatment beam emission gate 18. Emission of the treatment beam B from the treatment beam emission gate 18 is controlled, for example, by a treatment beam irradiation controller (not shown). In the treatment system 1, the treatment beam emission gate 18 is an example of an “irradiator” in the claims.

In a treatment room in which the treatment system 1 is installed, three-dimensional coordinates of a reference position are set in advance as shown in FIG. 1. In the treatment room in which a patient P is irradiated with a treatment beam B, an installation position of the treatment beam emission gate 18, a direction in which a patient P is irradiated with a treatment beam B (an irradiation direction), an installation position of the bed 12, an installation position of the CT imaging device 16, an imaging position of a CT image obtained by imaging the internal body of the patient P, and the like are ascertained on the basis of the three-dimensional coordinates of a preset reference position. In the following description, a three-dimensional coordinate system of the reference position preset in the treatment room is defined as a “room coordinate system.” In the following description, a “position” is defined as coordinates in three-axes directions (three dimensions) based on the translation mechanism provided in the bed 12 and expressed in the room coordinate system, and a “posture” is defined as rotation angles around the three axes based on the rotation mechanism provided in the bed 12 and expressed in the room coordinate system. For example, the position of the bed 12 is a position of a predetermined point in the bed 12 which is expressed as three-dimensional coordinates, and the posture of the bed 12 is rotation angles of the bed 12 which are expressed in yaw, roll, and pitch.

In radiation treatment, a treatment plan can be made in a situation in which the treatment room is simulated. That is, in radiation treatment, a state in which a patient P is placed on the bed 12 in the treatment room is simulated, and the irradiation direction, the irradiation intensity, and the like at the time of irradiation of the patient P with a treatment beam B are planned. Accordingly, information such as parameters indicating the position and the posture of the bed 12 in the treatment room is given to a CT image in a stage of treatment planning (treatment planning stage). This is the same in a CT image captured immediately before radiation treatment is performed or a CT image captured at the time of previous radiation treatment. That is, parameters indicating the position and the posture of the bed 12 at the time of imaging are given to a CT image obtained by imaging the internal body of the patient P using the CT imaging device 16.

In FIG. 1, the configuration of the treatment device 10 including the CT imaging device 16 and one fixed treatment beam emission gate 18 is shown, and the configuration of the treatment device 10 is not limited to this configuration. For example, the treatment device 10 may include an imaging device that generates a three-dimensional image of an internal body of a patient P such as a CT imaging device with a configuration in which a set of a radiation source and a radiation detector rotates inside of the ring-shaped opening, a cone-beam (CB) CT device, a magnetic resonance imaging (MRI) device, or an ultrasonic diagnosis device instead of the CT imaging device 16. For example, the treatment device 10 may have a configuration including a plurality of treatment beam emission gates in which a treatment beam emission gate irradiating a patient P with a treatment beam in the horizontal direction is additionally provided. For example, the treatment device 10 may have a configuration in which one treatment beam emission gate 18 shown in FIG. 1 rotates around the patient P such as rotates by 360 degrees about a rotation shaft in the horizontal direction X shown in FIG. 1 and irradiates the patient P with a treatment beam in various directions. For example, the treatment device 10 may include one or more imaging devices including a set of a radiation source and a radiation detector instead of the CT imaging device 16, and the imaging device captures an image of the internal body of the patient P in various directions by rotating by 360 degrees about the rotation shaft in the horizontal direction X shown in FIG. 1. This configuration is referred to as a rotary gantry type treatment device. In this case, for example, one treatment beam emission gate 18 shown in FIG. 1 may rotate simultaneously about the same rotation shaft as the imaging device.

The medical image processing device 100 performs a process of aligning a position of a patient P at the time of performing radiation treatment on the basis of a CT image output from the CT imaging device 16. More specifically, the medical image processing device 100 performs a process of aligning a position of a tumor or tissue in the body of the patient P, for example, on the basis of a CT image of the patient P captured before radiation treatment is performed such as in the treatment planning stage or a current image of the patient P captured by the CT imaging device 16 in the treatment stage (treatment stage) in which radiation treatment is performed. Then, the medical image processing device 100 outputs a movement amount signal for moving the bed 12 to the bed controller 14 in order to align the irradiation direction of a treatment beam B emitted from the treatment beam emission gate 18 with a direction set in the treatment planning stage. That is, the medical image processing device 100 outputs a movement amount signal for moving the patient P in a direction in which the tumor or tissue subjected to treatment in the radiation treatment is appropriately irradiated with the treatment beam B to the bed controller 14.

The medical image processing device 100 and the bed controller 14 or the CT imaging device 16 of the treatment device 10 may be connected to each other in a wired manner or may be connected to each other in a wireless manner such as a local area network (LAN) or a wide area network (WAN).

### (Treatment Planning)

Treatment planning which is performed before a movement amount calculating process is performed in the medical image processing device 100 will be described below. In the treatment planning, an amount of energy, an irradiation direction, and a shape of an irradiation range of a treatment beam B (radiation) with which a patient P is irradiated, distribution proportions of doses when the treatment beam B is emitted a plurality of times, and the like are determined. More specifically, first, a planner of a treatment plan (such as a doctor) designates a boundary between an area of a tumor (a lesion) and an area of normal tissue, a boundary between the tumor and vital organs located in the vicinity thereof, and the like in a first image captured in the treatment planning stage (for example, a CT image captured by the CT imaging device 16). Then, in the treatment planning, an irradiation direction of a treatment beam B (a route along which the treatment beam B passes), an irradiation intensity, and the like are determined on the basis of a depth from a body skin of the patient P to a position of a tumor and a size of the tumor which are calculated from information on the tumor designated by the planner of the treatment plan (such as a doctor).

Designation of a boundary between an area of a tumor and an area of normal tissue corresponds to designation of a position and a volume of a tumor. The volume of a tumor is referred to as a gross tumor volume (GTV), a clinical target volume (CTV), an internal target volume (ITV), a planning target volume (PTV), or the like. The GTV is a volume of a tumor which can be visually ascertained from an image and is a volume which needs to be irradiated with a treatment beam B with a sufficient dose in radiation treatment. The CTV is a volume including the GTV and a latent tumor to be treated. The ITV is a volume obtained by adding a predetermined margin to the CTV in consideration of movement of the CTV due to physiological movement of the patient P which is predicted, or the like. The PTV (which is an example of an “irradiation field”) is a volume obtained by adding a margin to the ITV in consideration of an error in alignment of the patient P which is performed at the time of performing treatment. These volumes satisfy the relationship of Expression (1).

$$\begin{matrix}[ Math . 1 ] & \\ GTV ∈ CTV ∈ ITV ∈ PTV & ( 1 )\end{matrix}$$

On the other hand, a volume of vital organs located in the vicinity of a tumor in which radiation sensitivity is high and an influence of a dose of emitted radiation appears strongly is referred to as an organ at risk (OAR). A planning organ-at-risk volume (PRV) is designated as a volume obtained by adding a predetermined margin to the OAR. The PRV is designated by adding a volume (an area) which is irradiated with radiation to avoid the OAR which is not intended to destroy with the radiation as a margin. These volumes satisfy the relationship of Expression (2).

$$\begin{matrix}[ Math . 2 ] & \\ OAR ∈ PRV & ( 2 )\end{matrix}$$

In the treatment planning stage, a direction (a route) or an intensity of a treatment beam B (radiation) with which a patient P is irradiated is determined on the basis of a margin in consideration of an error which is likely to occur in actual treatment.

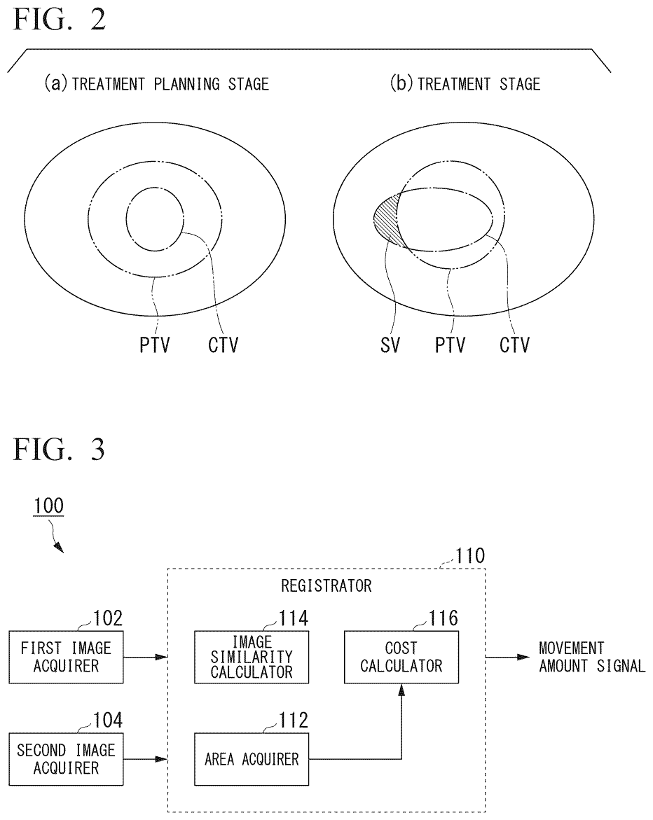

FIG. 2 is a diagram showing the CTV and the PTV in a treatment planning stage and a treatment stage. FIG. 2(a) shows the CTV and the PTV in the treatment planning stage, and FIG. 2(b) shows the CTV and the PTV in the treatment stage. In the treatment planning stage, a planner of a treatment plan designates a boundary between the CTV and the PTV in a CT image (more specifically, designates a boundary between the CTV and the PTV in a plurality of two-dimensional tomographic images obtained by cutting the CT image in a plurality of directions and converts the CTV and the PTV to three-dimensional CTV and PTV). The CTV and the PTV designated in the treatment planning stage are copied to a CT image captured in the treatment stage using a technique such as a deformable image registration (DIR) or an optical flow which will be described later. The CTV and the PTV in the treatment stage shown in FIG. 2(b) are obtained by copying the CTV and the PTV designated in the treatment planning stage shown in FIG. 2(a). At the time of treatment, the copied PTV is irradiated with the treatment beam B.

However, as indicated by reference sign SV in FIG. 2(b), an error of the CTV or the PTV accompanying the copying may be great and the CTV may depart from the PTV (inter-fraction) according to the type of an organ. In this case, the area SV departs from the irradiation range of the treatment beam B and causes a problem in treatment of a tumor. The medical image processing device 100 according to the present invention copes with this problem.

### (Configuration of Medical Image Processing Device)

A medical image processing device 100 according to a first embodiment will be described below. FIG. 3 is a block diagram schematically showing the medical image processing device 100 according to the first embodiment. The medical image processing device 100 includes, for example, a first image acquirer 102, a second imaging acquirer 104, and a registrator 110. The registrator 110 includes, for example, an area acquirer 112, an image similarity calculator 114, and a cost calculator 116.

Some or all of constituents of the medical image processing device 100 are realized, for example, by causing a hardware processor such as a central processing unit (CPU) to execute a program (software). Some or all of the constituents may be realized by hardware (a circuit unit which includes circuitry) such as a large scale integration (LSI) circuit, an application-specific integrated circuit (ASIC), a field-programmable gate array (FPGA), or a graphics processing unit (GPU) or may be cooperatively realized by software and hardware. Some or all functions of the constituents may be realized by a dedicated LSI circuit. The program may be stored in a storage device (a storage device including a non-transitory storage medium) such as a read only memory (ROM), a random access memory (RAM), a hard disk drive (HDD), or a flash memory provided in the medical image processing device 100 in advance or may be stored in a removable storage medium (a non-transitory storage medium) such as a DVD or a CD-ROM and installed in the HDD or the flash memory provided in the medical image processing device 100 by setting the storage medium into a drive device of the medical image processing device 100. The program may be downloaded from another computer device via a network and installed in the HDD or the flash memory of the medical image processing device 100.

The first image acquirer 102 acquires a first image associated with a patient P before treatment and parameters indicating a position and a posture at the time of capturing the first image. The first image is a three-dimensional CT image showing a three-dimensional shape of an internal body of a patient P which is captured, for example, by the CT imaging device 16 in the treatment planning stage at the time of performing radiation treatment. The first image is used to determine a direction (a route including a tilt or a distance) or an intensity of a treatment beam B with which the patient P is irradiated in radiation treatment. The determined direction (irradiation direction) or intensity of the treatment beam B is set in the first image. The first image is captured in a state in which the position and the posture (hereinafter referred to as a “posture”) of the patient P is kept constant by fixing the bed 12. Parameters indicating the posture of the patient P at the time of capturing the first image may be a position or a posture (an imaging direction or an imaging magnification) of the CT imaging device 16 at the time of capturing the first image or may be, for example, a position and a posture of the bed 12 at the time of capturing the first image, that is, set values set in the translation mechanism and the rotation mechanism provided in the bed 12 to keep the posture of the patient P constant. The first image acquirer 102 outputs the acquired first image and the acquired parameters to the registrator 110.

The second imaging acquirer 104 acquires a second image associated with the patient P which is captured immediately before radiation treatment is started and parameters indicating the position and the posture at the time of capturing the second image. The second image is a three-dimensional CT image showing a three-dimensional shape of the internal body of the patient P which is captured, for example, by the CT imaging device 16 in order to align the posture of the patient P at the time of irradiation with the treatment beam B in the radiation treatment. That is, the second image is an image which is captured by the CT imaging device 16 in a state in which the treatment beam B is not emitted from the treatment beam emission gate 18. In other words, the second image is a CT image which is captured at a time point different from the time point at which the first image has been captured. In this case, the first image and the second image are different from each other in imaging time point, but are equal to each other in an imaging method. Accordingly, the second image is captured in almost the same posture as the posture at the time of capturing the first image. Parameters indicating the posture of the patient P at the time of capturing the second image may be a position or a posture (an imaging direction or an imaging magnification) of the CT imaging device 16 at the time of capturing the second image or may be, for example, a position and a posture of the bed 12 at the time of capturing the second image, that is, set values set in the translation mechanism and the rotation mechanism provided in the bed 12 to make the posture of the patient P approach the same posture as the posture at the time of capturing the first image. The second imaging acquirer 104 outputs the acquired second image and the acquired parameters to the registrator 110.

The first image and the second image are not limited to CT images captured by the CT imaging device 16 and may be three-dimensional images captured by an imaging device other than the CT imaging device 16 such as a CBCT device, an MRI device, or an ultrasonic diagnosis device. For example, the first image may be a CT image, and the second image may be a three-dimensional image captured by an MRI device.

The first image and the second image may be a two-dimensional image such as an X-ray transparent image. In this case, the first image acquirer 102 and the second imaging acquirer 104 may acquire DRR images obtained by virtually reconstructing a transparent image from a three-dimensional CT image and use the DRR images as the first image and the second image. When the first image and the second image are two-dimensional images, the parameters indicating the position and the posture are a position in the treatment room in the image and a rotation angle in a plane.

### (Generation of Integral Image)

When the first image and the second image are aligned, for example, a spatial position with a high image similarity (for example, a high difference in pixel value between the first image and the second image) is calculated while fixing the position and the posture of the second image disposed in the room coordinate system and moving the first image. However, in this method, the difference in pixel value between the first image and the second image decreases, but it cannot be said that a dose distribution of a treatment beam B for a tumor designated by a planner (such as a doctor) in a treatment plan important in radiation treatment is calculated to match each other. Since radiation (a treatment beam B herein) loses energy at the time of passing through substance, a radiation irradiation method is determined by calculating an amount of energy loss of a virtually emitted radiation using a CT Image in the treatment planning. In consideration of this, when a position of a patient P is aligned in the treatment stage, it is important to align tissue in the body of the patient P located on a route along which the emitted treatment beam B passes.

In consideration of these circumstances, in order to enable alignment for causing energy applied to a tumor in the body of a patient P by an emitted treatment beam B to approach energy planned in the treatment planning stage, the first image acquirer 102 and the second imaging acquirer 104 generate integral images (water-equivalent thickness images) by integrating pixel values (CT values) of pixels (voxels) located on a route along which the treatment beam B passes in CT images and acquire the generated integral images as a first image and a second image. That is, the first image acquirer 102 and the second imaging acquirer 104 also serve as an “image converter” in the claims. The first image acquirer 102 and the second imaging acquirer 104 output the first image and the second image which are the generated integral images to the registrator 110. A method of calculating an integral image will be schematically described below, for example, using the first image acquirer 102 that calculates a first integral image corresponding to the first image which is a CT image.

In calculation of a first integral image in the first image acquirer 102, first, pixels located on a route along which the treatment beam B passes are extracted out of pixels included in the first image. Regarding the route along which the treatment beam B passes, a route along which the treatment beam B emitted from the treatment beam emission gate 18 passes through the patient P can be acquired as three-dimensional coordinates in the room coordinate system on the basis of the irradiation direction of the treatment beam B included in information on directions in the treatment room (hereinafter referred to as “direction information”). The direction information includes, for example, information indicating the irradiation direction of the treatment beam B and information indicating a movement direction of the bed 12. The direction information is information which is expressed in a preset room coordinate system. The route along which the treatment beam B passes may be acquired as a three-dimensional vector with the position of the treatment beam emission gate 18 indicated by three-dimensional coordinates in the room coordinate system as a start point.

The first image acquirer 102 calculates the first integral image by integrating pixel values (CT values) of pixels (voxels) located on the route along which the treatment beam B passes in the first image on the basis of the first image output from the first image acquirer 102, the parameters indicating the position and the posture of the first image, and the direction information.

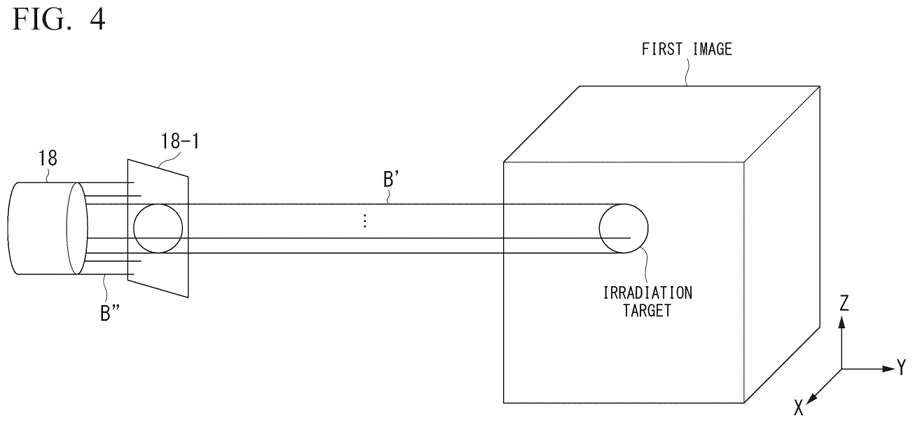

The irradiation direction of a treatment beam B emitted from the treatment beam emission gate 18 will be described below. In the following description, it is assumed that the route of a treatment beam B is a three-dimensional vector. FIG. 4 is a diagram showing an example of a relationship between emission of radiation (a treatment beam B) and an irradiation target (a tumor in the body of a patient P) of the radiation (the treatment beam B) in the treatment system 1. In FIG. 4, an example of a route along which a treatment beam B emitted from the treatment beam emission gate 18 reaches an area (a range) of a tumor in the body of the patient P which is an irradiation target is shown. FIG. 4 shows an example of a configuration in which a treatment beam B is emitted from the treatment beam emission gate 18.

In the configuration in which the treatment beam emission gate 18 emits a treatment beam B, the treatment beam emission gate 18 includes a planar emission port as shown in FIG. 4. The treatment beam B emitted from the treatment beam emission gate 18 reaches a tumor which is an irradiation target via a collimator 18-1. That is, only a treatment beam B′ passing through the collimator 18-1 out of the treatment beam B emitted from the treatment beam emission gate 18 reaches a tumor which is the irradiation target. The collimator 18-1 is a metallic mechanism for blocking an unnecessary treatment beam B″. In the collimator 18-1, an area through which the treatment beam B passes is adjusted, for example, according to a shape of the tumor which is the irradiation target such that an area other than the tumor in the body of the patient P is not irradiated with the treatment beam B. For example, the collimator 18-1 may be a multi-leaf collimator that can mechanically change the area for blocking the unnecessary treatment beam B″. In FIG. 4, an example in which a tumor which is an irradiation target in the first image is irradiated with the treatment beam B′ passing through the collimator 18-1 out of the treatment beam B is schematically shown. In this case, a start point of the route of the treatment beam B′is a position of an emission point of the treatment beam B′ located in a range of the planar emission port of the treatment beam emission gate 18. The three-dimensional position of the treatment beam emission gate 18 is, for example, a position (coordinates) of the planar center of the emission port.

The first image acquirer 102 acquires direction information including an irradiation direction of the treatment beam B′ as information indicating the irradiation direction of the treatment beam B. The first image acquirer 102 uses a route along which the treatment beam B′ reaches a tumor which is an irradiation target in the first image as the route of the treatment beam B′ which is emitted in a predetermined three-dimensional space. Here, the tumor position of the irradiation target can be expressed by position i in the room coordinate system, and the route b(i) of the treatment beam B′ reaching the position can be discretely expressed by a set of three-dimensional vectors as expressed by Expression (3).

$$[ Math . 3 ]$$

$$B = \{ {b}_{i} / i ∈ Ω \} \qquad ( 3 )$$

A start point of each route, that is, a start point of a three-dimensional vector b(i), is a position of the emission point of the treatment beam B′ reaching the tumor which is the irradiation target on the route b(i). A three-dimensional position of the start point is defined as S. Ω denotes a set of positions in the room coordinate system of the tumor position as the irradiation target, that is, the PTV or the GTV.

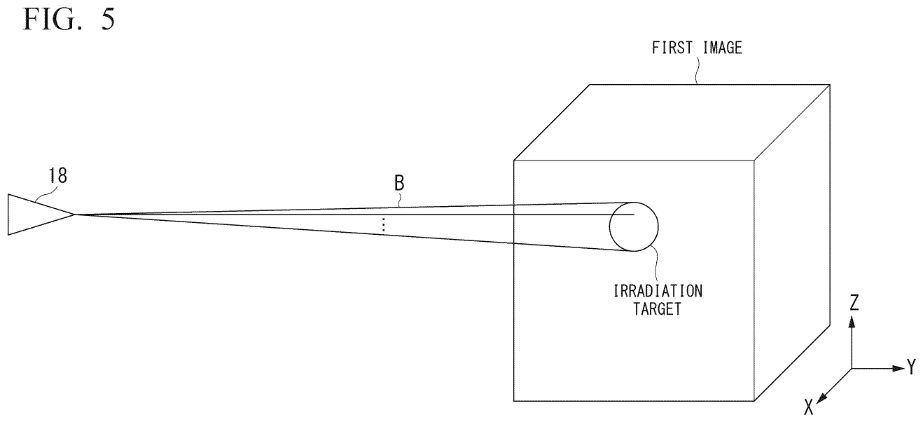

FIG. 5 is a diagram showing another example of a relationship between emission of radiation and an irradiation target of the radiation in the treatment system. In FIG. 5, an example of a route along which a treatment beam B emitted from the treatment beam emission gate 18 reaches an area (a range) of a tumor in the body of the patient P which is an irradiation target is shown. FIG. 5 shows an example in which scanning with the treatment beam B emitted from the treatment beam emission gate 18 is performed. In this configuration, the treatment beam emission gate 18 does not include the collimator 18-1 and includes an emission port as shown in FIG. 5. A treatment beam B emitted from one emission port of the treatment beam emission gate 18 is bent in direction, for example, by a magnet or the like, scans to cover the whole area of a tumor which is an irradiation target, and is applied to the tumor which is an irradiation target. In FIG. 5, an example in which the irradiation direction of the treatment beam B is scanned and the treatment beam B is applied to the tumor which is an irradiation target in the first image is schematically shown. In this case, a start point of each route of the scanning treatment beam B is the position of the emission port of the treatment beam emission gate 18. The three-dimensional position of the treatment beam emission gate 18 is the position (coordinates) of the one emission port. In this case, the route b(i) of the treatment beam B reaching a certain position i in the room coordinate system can be discretely expressed as expressed by Expression (3).

The first image acquirer 102 acquires direction information including the irradiation direction in which scanning with the treatment beam B is performed as information indicating the irradiation direction of the treatment beam B and sets a route b(i) along which the emitted treatment beam B reaches the coordinates i in the room coordinate system indicating the position of the tumor which is an irradiation target in the first image as a route of the treatment beam B emitted into a predetermined three-dimensional space. The route of the treatment beam B in this case can also be discretely expressed by a set of three-dimensional vectors as expressed by Expression (3). A start point of each route, that is, a start point of the three-dimensional vector b(i), is the position of the emission port of the treatment beam emission gate 18.

A method of calculating an integral image on the basis of the set route of the treatment beam B will be described below. In the following description, a position i of a point in a predetermined three-dimensional space (the room coordinate system) is defined as a point i. A pixel value of a three-dimensional pixel corresponding to the point i included in the first image which is virtually disposed in the predetermined three-dimensional space is defined as Iᵢ(x). Similarly, a pixel value of a three-dimensional pixel corresponding to the point i included in the second image which is virtually disposed in the predetermined three-dimensional space is defined as Tᵢ(x). A pixel value when there is no pixel corresponding to the point i in the first image or the second image is defined as “0.” Here, x is a parameter of a vector x indicating a position and a posture of the first image or the second image in the predetermined three-dimensional space.

Regarding the treatment beam B, a vector from a three-dimensional vector 0 of the position of the emission port of the treatment beam emission gate 18, that is, a start point S, to the point i can be expressed by Expression (4).

$$[ Math . 4 ]$$

$$b ( i ) = i - 0 \qquad ( 4 )$$

In this case, Expression (5) of a pixel value of a pixel included in a first integral image (hereinafter referred to as an “integral pixel value”) which is obtained by the first image acquirer 102 integrating pixel values of pixels located on the route of the treatment beam B to the point i in the first image can be defined as Expression (6).

$$[ Math . 5 ]$$

$${\tilde{I}}_{i} ( x ) \qquad ( 5 )$$

$$[ Math . 6 ]$$

$${\tilde{I}}_{i} ( x ) = {\int }_{0}^{1} f ( {I}_{i - tb ( i )} ( x ) ) dt \qquad ( 6 )$$

Similarly, Expression (7) of an integral pixel value which is obtained by the second imaging acquirer 104 integrating pixel values of pixels included in the second integral image located on the route of the treatment beam B to the point i in the second image can be defined as Expression (8).

$$[ Math . 7 ]$$

$${\tilde{R}}_{i} ( x ) \qquad ( 7 )$$

$$[ Math . 4 ]$$

$${\tilde{R}}_{i} ( x ) = {\int }_{0}^{1} f ( {R}_{i - lb ( i )} ( x ) ) dt \qquad ( 8 )$$

In Expression (6) and Expression (8), t is a parameter, and f(x) is a function for converting a pixel value (CT value) of a CT image. The function f(x) is, for example, a function based on a conversion table for converting an amount of energy loss of radiation to a water-equivalent thickness. As described above, radiation loses energy at the time of passing through substance. At this time, the amount of energy loss of the radiation is an amount of energy corresponding to the CT value of the CT image. That is, the amount of energy loss of radiation is not uniform and varies, for example, according to the tissue in the body of a patient P such as bones or fats. The water-equivalent thickness is a value indicating an amount of energy loss of radiation which varies depending on tissue (substance) as a thickness of water which is the same substance and can be obtained through conversion based on the CT value. For example, when the CT value is a value indicating a bone, the amount of energy loss of the radiation at the time of passing through the bone is large, and thus the water-equivalent thickness has a large value. For example, when the CT value is a value indicating a fat, the amount of energy loss of the radiation at the time of passing through the fat is small, and thus the water-equivalent thickness has a small value. For example, when the CT value is a value indicating air, the energy of the radiation is not lost at the time of passing through the air, and thus the water-equivalent thickness is “0.” By converting the CT values included in the CT image to the water-equivalent thicknesses, an amount of energy loss in each pixel located on the route of the treatment beam B can be expressed with the same criterion. As a conversion expression for converting a CT value to a water-equivalent thickness, for example, a regression expression based on nonlinear conversion data which is experimentally acquired is used. Various documents are published regarding the nonlinear conversion data which is experimentally acquired. The function f(x) may be, for example, a function for performing identity mapping. Alternatively, definition of the function f(x) may be changed according to a treatment site. As described above, the first image acquirer 102 and the second imaging acquirer 104 acquire the first image and the second image as integral images.

### (Estimation and Acquisition of Area)

The area acquirer 112 acquires two or more areas corresponding to one or both of the first image and the second image from the first image and the second image and outputs the acquired two or more areas to the cost calculator 116. More specifically, the area acquirer 112 acquires an area (such as a PTV or a CTV) including a position or a volume of a tumor designated in the treatment planning from the first image and acquires an area which is obtained by estimating movement of the area designated in the first image from the second image.

In order to estimate movement of an area, the area acquirer 112 calculates movement of an area in the second image similar to an image in the area of the tumor designated in the first image. As a method thereof, the area acquirer 112 uses, for example, techniques such as DIR or an optical flow.

As an example of the method of calculating an optical flow, the area acquirer 112 retrieves a position of a most similar image as the position of the tumor in the second image by setting an image indicating the area of the tumor designated in the first image as a template and performing template matching on the second image. Then, the area acquirer 112 calculates a motion vector of the found position of the tumor in the second image and uses all the calculated motion vectors as motion models. The area acquirer 112 may divide the area of the tumor used as the template into a plurality of small areas (hereinafter referred to as “subareas”) and use an image indicating each subarea as a template. In this case, the area acquirer 112 performs template matching for each template of the subareas and searches for a most similar position of the tumor in in the second image for each subarea. Then, the area acquirer 112 calculates a motion vector of the corresponding position of the tumor in the second image for each found subarea and uses all the calculated motion vectors as motion models. The area acquirer 112 may use a mean vector, a median vector, or the like of the calculated motion vectors as motion models.

As another example of the method of calculating an optical flow, the area acquirer 112 may calculate movement of an area in the second image similar to a distribution of pixel values in the area of the tumor designated in the first image. In this method, for example, the area acquirer 112 may use a technique of searching for a position having a similar histogram of pixel values through mean shift, medoid shift, or the like and tracking an object. At this time, the area acquirer 112 generates a motion model using a distribution of the histogram of pixel values calculated using all the pixel values in the area of the tumor designated in the first image. The area acquirer 112 may divide the area of the tumor designated in the first image into a plurality of subareas and generate a motion model corresponding to each subarea using the distribution of the histogram of pixel values calculated using the pixel values in the area for each subarea. In this case, the area acquirer 112 may set a plurality of motion models corresponding to each subarea as a motion model group or set a mean vector, a median vector, or the like of the motion model group as a motion model.

The area acquirer 112 may set an area acquired from the first image to an area equal to or smaller than a PTV set in the second image. Accordingly, the area acquired from the first image can be reliably included in the PTV of the second image. The area acquired by the area acquirer 112 is not all the areas determined in treatment planning, but may be some areas such as a PTV or an OAR.

### (Calculation of Image Similarity)

The image similarity calculator 114 acquires the first image and parameters indicating the position and the posture thereof from the first image acquirer 102, acquires the second image and parameters indicating the position and the posture thereof from the second imaging acquirer 104, calculates an image similarity between the first image and the second image, and outputs the calculated image similarity to the registrator 110. More specifically, for example, the image similarity calculator 114 calculates an absolute value of a difference in pixel value at the same spatial position between the first image and the second image using Expression (9) and calculates the total sum in the whole image as the image similarity.

$$[ Math . 9 ]$$

$${\vert I ( x + Δ x ) - R ( {x}_{plan} ) \vert}^{2} \qquad ( 9 )$$

In Expression (9), Δx denotes an amount of misalignment in position and posture between the first image and the second image, xₚₗₐₙ denotes coordinates included in a PTV in the treatment plan, and R(xₚₗₐₙ) denotes a pixel value in the coordinates xₚₗₐₙ.

The image similarity calculator 114 may use normalized cross-correlation at the same spatial position between the first image and the second image as the similarity. At this time, a range in which correlation is taken is a subarea of 3×3×3 or the like from a pixel of a calculation target. The image similarity calculator 114 may use an amount of mutual information at the same spatial position between the first image and the second image as the similarity. In calculating the similarity, the image similarity calculator 114 may limit the calculation to pixels in an area corresponding to each image.

### (Calculation of Cost)

In radiation treatment, when a tumor departs from a PTV, there is a risk that a dose planned in the treatment plan may not be met and a sufficient treatment effect may not be obtained. When a dose equal to or greater than planned is applied to an OAR, there is a risk that side effects may increase. These risks cannot be measured on the basis of the image similarity. Therefore, an index called a “cost” for calculating such a risk is introduced.

The cost calculator 116 calculates a cost based on a positional relationship between areas using two or more areas acquired from the area acquirer 112 and outputs the calculated cost to the registrator 110. More specifically, the cost calculator 116 calculates the cost based on the positional relationship between areas using Expression (10).

$$[ Math . 10 ]$$

$$λ {f}_{0} ( x + Δ x ) \qquad ( 10 )$$

In Expression (10), f₀ denotes a cost function, and λ denotes a weight given to the cost. For example, the cost function f₀ is defined to have a larger value as the CTV in the treatment stage departs more from the range of the PTV as shown in FIG. 2(b) and to be reflected as a cost. In this case, since the CTV in the treatment stage protrudes externally from the PTV, there is a likelihood that the dose for a tumor may become lower than planned and a treatment effect may decrease. Therefore, a value based on a volume (SV) (or an area) of the protruding part is defined as the cost function f₀.

Alternatively, the cost function f₀ may be designed to increase as the PTV and the OAR in the first image becomes closer in position. In this way, it is possible to design the cost function on the basis of the positional relationship between two or more areas determined in the treatment planning. The cost function f₀ may be defined as a linear sum of a plurality of cost functions. For example, the weight λ may increase as the PTV narrows more or increase as the PTV and the OAR become closer in position.

### (Execution of Registration)

The registrator 110 calculates a position of the first image such that the calculated image similarity increases and the cost decreases on the basis of the calculation result from the image similarity calculator 114 and the calculation result from the cost calculator 116. More specifically, first, the registrator 110 defines the cost function E(Δx) as a sum of the calculation result from the image similarity calculator 114 and the calculation result from the cost calculator 116 as expressed by Expression (11).

$$[ Math . 11 ]$$

$$E ( Δ x ) = {\vert I ( x + Δ x ) - R ( {x}_{plan} ) \vert}^{2} + λ {f}_{0} ( x + Δ x ) \qquad ( 11 )$$

By performing Taylor expansion of Expression (11) with respect to x, Expression (12) is obtained.

$$[ Math . 12 ]$$

$$E ( Δ x ) = {\vert I ( x ) + {∇}_{I}^{T} ( x ) Δ x - R ( {x}_{plan} ) \vert}^{2} + λ ( {f}_{0} ( x ) + {∇}_{{f}_{0}}^{T} ( x ) Δ x ) \qquad ( 12 )$$

In order to calculate a minimum value of the amount of misalignment Δx, Expression (13) is obtained by differentiating the right side with respect to an amount of movement Δx to set the right side to 0.

$$[ Math . 13 ]$$

$$\frac{\partial E ( Δ x )}{\partial Δ x} = 2 ( I ( x ) + {∇}_{I}^{T} ( x ) Δ x - R ( {x}_{plan} ) ) {∇}_{I} ( x ) + λ {∇}_{{f}_{0}} ( x ) = 0 \qquad ( 13 )$$

Expression (14) is obtained by solving Expression (13) with respect to Δx.

$$[ Math . 14 ]$$

$$Δ x = {H}^{- 1} ( {∇}_{I} ( x ) ( R ( {x}_{plan} ) - I ( x ) ) - \frac{λ}{2} {∇}_{{f}_{0}} ( x ) ) \qquad ( 14 )$$

In Expression (14), H is a hessian matrix which is defined by Expression (15). In Expression (15), V denotes a vector of a position and a posture when the first image is disposed in a predetermined three-dimensional space. The vector V has the same number of dimensions as the number of axes indicated by the direction information and is, for example, a six-dimensional vector in case of the six degrees of freedom.

$$[ Math . 15 ]$$

$$H = {\sum }_{i ∈ Ω} {∇}_{i} ( V ) {∇}_{i}^{T} ( V ) \qquad ( 15 )$$

The registrator 110 calculates the cost function E(Δx) by substituting coordinate xₚₗₐₙ indicating a candidate position which is prepared in advance as an initial value of x in Expression (11) and calculates Δx using Expression (14). Then, the registrator 110 updates x with x=x+Δx using the calculated Δx and recalculates the cost function E(Δx). The registrator 110 ends this process when calculation of the cost function E(Δx) is repeated a predetermined number of times or a difference between the previous result and the present result of the cost function E(Δx) is less than a threshold value. The registrator 110 calculates an amount of movement of the bed 12 on the basis of the amount of misalignment Δx at a time point at which the process ends and outputs a movement amount signal.

In the above description, the registrator 110 defines the cost function E(Δx) using Expression (11). However, the present invention is not limited thereto, and the registrator 110 may define the cost function E(Δx), for example, using Expression (16).

$$[ Math . 16 ]$$

$$E ( Δ x ) = {\vert I ( x + Δ x ) - R ( {x}_{plan} ) \vert}^{2} + {λ}_{1} {f}_{0} ( x + Δ x ) + {λ}_{2} {\vert Δ x \vert}^{2} \qquad ( 16 )$$

In Expression (16), λ₂|Δx|² is added to the cost function E(Δx) of Expression (14). In Expression (16) λ₁ corresponds to λ in Expression (12), and λ₂ denotes a weight which is applied to the amount of misalignment |Δx|². That is, by defining the cost function E(Δx) using Expression (16), it is possible to calculate the cost in consideration of the amount of misalignment between CT images.

By performing Taylor expansion of Expression (16) with respect to x, Expression (17) is obtained.

$$[ Math . 17 ]$$

$$( 17 )$$

$$E ( Δ x ) = {\vert I ( x ) + {∇}_{1}^{T} ( x ) Δ x - R ( {x}_{plan} ) \vert}^{2} + {λ}_{1} ( {f}_{0} ( x ) + {∇}_{{f}_{0}}^{T} ( x ) Δ x ) + {λ}_{2} {\vert Δx \vert}^{2}$$

In order to calculate the minimum value of the amount of misalignment Δx, Expression (18) is obtained by differentiating the right side with respect to the amount of movement Δx to set the right side to 0.

$$[ Math . 18 ]$$

$$\frac{\partial E ( Δ x )}{\partial Δ x} = 2 ( I ( x ) + {Δ}_{I}^{T} ( x ) Δ x - R ( {x}_{plan} ) ) {∇}_{I} ( x ) + {λ}_{1} {∇}_{{f}_{0}} ( x ) + 2 {λ}_{2} Δ x = 0 \qquad ( 18 )$$

Expression (19) is obtained by solving Expression (18) with respect to Δx.

$$[ Math . 19 ]$$

$$Δ x = {H}_{2}^{- 1} ( {∇}_{I} ( x ) ( R ( {x}_{plan} ) - I ( x ) ) - \frac{λ}{2} {∇}_{J = {f}_{0}} ( x ) ) \qquad ( 19 )$$

In Expression (19), H₂ is a hessian matrix which is defined by Expression (20). By adding λ₂|Δx|2 to the cost function E(Δx) of Expression (16), λ₂E is added as a remainder term to H₂ unlike the hessian matrix H of Expression (15).

$$\begin{matrix}[ Math . 20 ] & \\ {H}_{2} = {Σ}_{i ∈ Ω} {∇}_{i} ( V ) {∇}_{i}^{T} ( V ) + {λ}_{2} E & ( 20 )\end{matrix}$$

The registrator 110 calculates the cost function E(Δx) by substituting coordinates xₚₗₐₙ indicating a candidate position which is prepared in advance as an initial value of x in Expression (16) and calculates Δx using Expression (19). Then, the registrator 110 updates x with x=x+Δx using the calculated Δx and recalculates the cost function E(Δx). The registrator 110 ends this process when calculation of the cost function E(Δx) is repeated a predetermined number of times or a difference between the previous result and the present result of the cost function E(Δx) is less than a threshold value. The registrator 110 calculates an amount of movement of the bed 12 on the basis of the amount of misalignment Δx at a time point at which the process ends and outputs a movement amount signal.

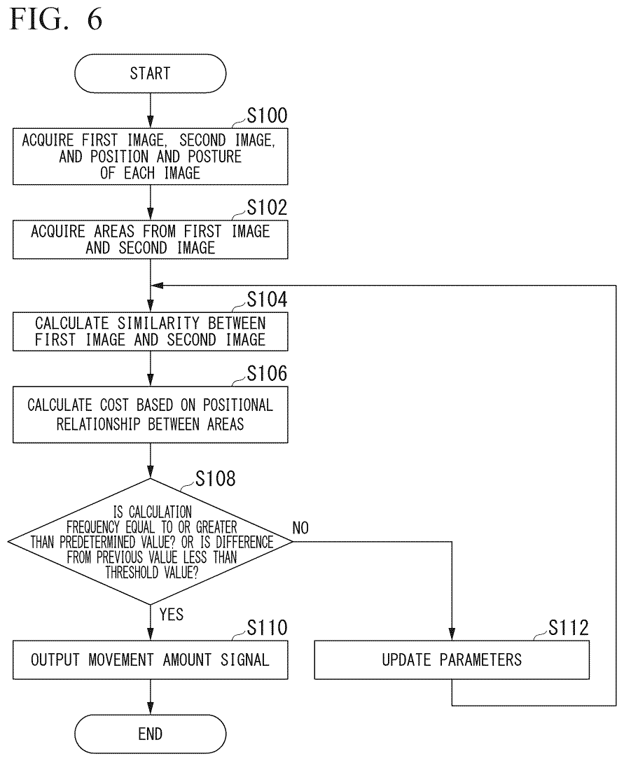

A process flow that is performed by the medical image processing device 100 according to the first embodiment will be described below with reference to FIG. 6. FIG. 6 is a flowchart showing a process flow that is performed by the medical image processing device 100 according to the first embodiment.

First, the medical image processing device 100 acquires a first image and parameters indicating a position and a posture thereof and a second image and parameters indicating a position and posture thereof using the first image acquirer 102 and the second imaging acquirer 104 (Step S100). Then, the medical image processing device 100 acquires two or more areas corresponding to one or both of the first image and the second image using the area acquirer 112 (Step S102).

Then, the medical image processing device 100 calculates a similarity between the first image and the second image using the image similarity calculator 114 (Step S104). Then, the medical image processing device 100 calculates a cost on the basis of the positional relationship between the acquired two or more areas using the cost calculator 116 (Step S106).

Then, the medical image processing device 100 calculates a sum of the similarity and the cost using the registrator 110 and determines whether the number of times of calculation is equal to or greater than a predetermined number of times or a difference between the present value and the previous value of the calculated sum is less than a threshold value (Step S108). When it is determined that the number of times of calculation is less than the predetermined number of times or the difference between the present value and the previous value of the calculated sum is less than the threshold value, the medical image processing device 100 calculates and outputs a movement amount signal corresponding to the amount of misalignment Δx using the registrator 110 (Step S110). On the other hand, when it is determined that the number of times of calculation is not less than the predetermined number of times and the difference between the present value and the previous value of the calculated sum is greater than the threshold value, the medical image processing device 100 updates the parameter x with x=x+Δx using the registrator 110 and returns the process flow to Step S104. Accordingly, the process flow of the flowchart ends.

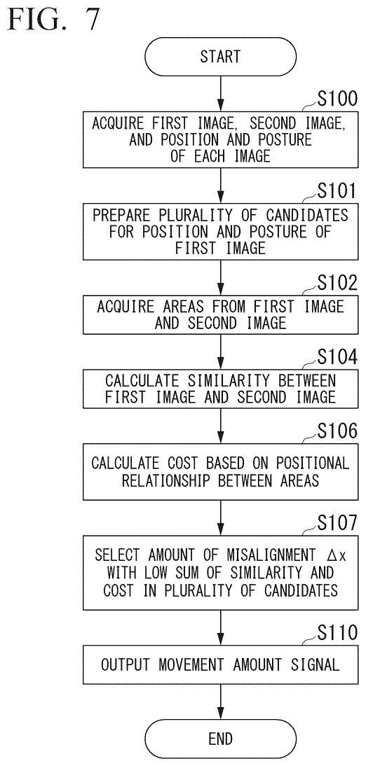

In the flowchart, the registrator 110 determines whether the number of times of calculation is less than the predetermined number of times or the difference between the present value and the previous value of the calculated sum is less than the threshold value. However, the present invention is not limited to this configuration. FIG. 7 is a flowchart showing another example of the process flow that is performed by the medical image processing device 100 according to the first embodiment. Differences from the process flow of the flowchart shown in FIG. 6 will be mainly described below.

When the first image acquirer 102 acquires a first image in Step S100, the first image acquirer 102 prepares a plurality of candidates for the position and the posture of the first image (Step S101). In Step S104, the image similarity calculator 114 calculates a similarity for each of the prepared plurality of candidates. In Step S106, the cost calculator 116 calculates a cost for each of the prepared plurality of candidates. In Step S107, the registrator 110 selects an amount of misalignment Δx in which the sum of the similarity and the cost is a minimum out of the plurality of candidates and outputs a movement amount signal corresponding to the selected amount of misalignment Δx (Step S110). Accordingly, the process flow of the flowchart ends.

According to the first embodiment described above, when CT images of a patient captured at the time of treatment planning and at the time of treatment are aligned, the cost is calculated using a positional relationship among a tumor or an irradiation field at the time of treatment and an organ at risk or the like in addition to the similarity between the CT images and outputs a movement amount signal in which the similarity is high and the cost is low. Accordingly, it is possible to perform image comparison at a high speed and with high accuracy.

### Second Embodiment

A second embodiment will be described below. In the first embodiment, a method of calculating best position and posture out of positions and postures of candidates which are prepared in advance has been described above. However, in this method, the number of combinations becomes larger and the calculation time increases as the number of degrees in position and posture increases. For example, there are three parameters of an X axis, a Y axis, and a Z axis as parameters indicating a position in a three-dimensional space, and there are three parameters of rotation angles about the X, Y, and Z axes as parameters indicating a posture, and thus there are six parameters in total. In consideration of these circumstances, a medical image processing device 100B according to the second embodiment can calculate a position and a posture more efficiently than in the first embodiment.

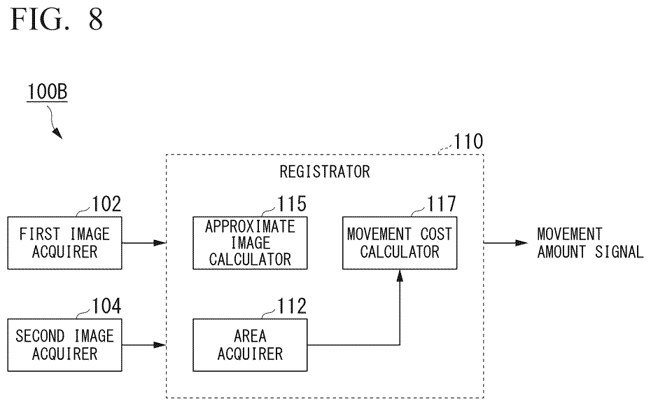

FIG. 8 is a block diagram schematically showing a configuration of the medical image processing device 100B according to the second embodiment. The medical image processing device 100B includes a first image acquirer 102, a second imaging acquirer 104, and a registrator 110. The registrator 110 includes an area acquirer 112, an approximate image calculator 115, and a movement cost calculator 117. Elements different from those in the first embodiment will be mainly described below.

The approximate image calculator 115 acquires a first image and parameters indicating a posture and a posture thereof, acquires a second image and parameters indicating a position and a posture thereof from the second imaging acquirer 104, and calculates an approximate image in the position and the posture of the first image. The approximate image calculator 115 outputs the calculated approximate image to the registrator 110. A method of specifically calculating an approximate image will be described below.

### (Calculation of Approximate Image)

In the following description, a pixel (voxel) included in the first image which is virtually disposed in a predetermined three-dimensional space based on the room coordinate system is defined as Iᵢ(V). In a pixel Iᵢ(V), Expression (19) represents a three-dimensional position in the room coordinate system.

$$\begin{matrix}[ Math . 21 ] & \\ i ∈ {ℝ}^{3} & ( 21 )\end{matrix}$$

A vector V may have a small number of dimensions corresponding to directions of the degrees of freedom when movement of the bed 12 is controlled. For example, when the number of directions of the degrees of freedom in which movement of the bed 12 is controlled is four, the vector V may be a four-dimensional vector. On the other hand, the number of dimensions of the vector V may be increased by setting the irradiation direction of the treatment beam B to the movement direction of the bed 12 on the basis of direction information output from the direction acquirer 106. For example, when the irradiation direction of the treatment beam B included in the direction information includes two directions of a vertical direction and a horizontal direction and the movement directions of the bed 12 includes six directions of the degrees of freedom, the vector V may be a vector of a total of eight dimensions.

The approximate image calculator 115 calculates an approximate image which is obtained by moving (translating and rotating) the first image by a minute amount of movement ΔV. Here, the amount of movement ΔV is a minute amount of movement which is preset as a parameter. The approximate image calculator 115 calculates (approximates) pixels Iᵢ(V+ΔV) included in the approximate image corresponding to the pixels Iᵢ(V) included in the first image using Expression (22) and Taylor expansion.

$$\begin{matrix}[ Math . 22 ] & \\ {I}_{i} ( V + Δ V ) = {I}_{i} ( V ) + {∇}_{i}^{T} ( V ) Δ V + ϵ & ( 22 )\end{matrix}$$

In Expression (22), ε of the third term on the right side is a term into which secondary or higher terms in the pixels Iᵢ(V+ΔV) are summarized and expressed. ∇ᵢ(V) is a value of a primary differential indicating a change of a vector which varies depending on degrees of freedom of a three-dimensional space covered by the vector V. ∇ᵢ(V) is expressed as a vector with the same number of dimensions as the vector V indicating a change in pixel value (for example, CT value) between the corresponding pixels located at the same position i in the room coordinate system in the first image before being moved (before being approximated) and the approximate image minutely moved. For example, when the movement direction of the bed 12 includes six directions of the degrees of freedom, the six-dimensional vector ∇ᵢ(V) corresponding to the pixel Iᵢ(V) located at the center position i in the room coordinate system in the first image is expressed by Expression (23).

$$\begin{matrix}[ Math . 23 ] & \\ {∇}_{i} ( V ) = \{ \begin{matrix}( {I}_{i} ( V + Δ {V}_{{θ}_{x}} ) - {I}_{i} ( V ) / Δ {θ}_{x} \\ ( {I}_{i} ( V + Δ {V}_{{θ}_{y}} ) - {I}_{i} ( V ) ) / Δ {θ}_{y} \\ ( {I}_{i} ( V + Δ {V}_{{θ}_{z}} ) - {I}_{i} ( V ) ) / Δ {θ}_{z} \\ ( {I}_{i} ( V + Δ {V}_{{t}_{x}} ) - {I}_{i} ( V ) ) / Δ {t}_{x} \\ ( {I}_{i} ( V + Δ {V}_{{t}_{y}} ) - {I}_{i} ( V ) ) / Δ {t}_{y} \\ ( {I}_{i} ( V + Δ {V}_{{t}_{z}} ) - {I}_{i} ( V ) / Δ {t}_{z}\end{matrix} \} \begin{matrix} \\ \\ \\ \\ \\ \end{matrix} & ( 23 )\end{matrix}$$

In Expression (23), Δθx, Δθy, and Δθz denote rotation angles about three axes in the room coordinate system when the three axes are set to the x axis, the y axis, and the z axis, and Δtx, Δty, and Δtz denote amounts of movement along the axes. Elements on the right side of Expression (23) denote pixel values at the positions i in the room coordinate system in the first image. For example, “Expression (24)” which is a first element on the right side of Expression (23) is a pixel value when the pixel Iᵢ(V) located at the position i in the room coordinate system in the first image is rotated about the x axis by the rotation angle Δθx. In this example, Expression (25) is expressed by Expression (26). The other elements on the right side of Expression (23) can also be expressed in this way, and detailed description of the elements will be omitted.

$$\begin{matrix}[ Math . 24 ] & \\ {I}_{i} ( V + Δ {V}_{{θ}_{x}} ) & ( 24 )\end{matrix}$$

$$\begin{matrix}[ Math . 25 ] & \\ Δ {V}_{{θ}_{x}} & ( 25 )\end{matrix}$$

$$\begin{matrix}[ Math . 26 ] & \\ Δ {V}_{{θ}_{x}} = {( Δ {θ}_{x} , 0 , 0 , 0 , 0 , 0 )}^{T} & ( 26 )\end{matrix}$$

The approximate image calculator 115 outputs the approximate image obtained by performing calculation of moving (translating and rotating) the first image by a minute amount of movement ΔV as described above to the registrator 110. When information indicating that there is a misalignment between the first image and the second image is output from the registrator 110, the approximate image calculator 115 calculates a new approximate image by moving (translating and rotating) the first image by a more minute amount of movement ΔV in the same way and outputs the calculated new approximate image to the registrator 110.

The movement cost calculator 117 acquires two or more areas from the area acquirer 112, calculates a movement cost based on the positional relationship between the areas, and outputs the calculated movement cost to the registrator 110. Here, the movement cost means a cost based on the positional relationship between the areas similarly to the first embodiment.

In the second embodiment, this cost is defined as a function fᵢ(V) depending on V indicating the position and the posture of the first image. Here, i denotes a position in the room coordinate system expressed by Expression (21), and V denotes a vector indicating the position and the posture when the first image is disposed in the room coordinate system. The position and the posture of the second image are fixed, and thus description thereof is omitted.

The movement cost calculator 117 converts the first image to an area image including pixel values in which flag information indicating whether each pixel is included in the area is embedded, correlates the area image with the position and the posture of the first image and the second image, and then calculates an approximate image thereof. More specifically, the movement cost calculator 117 calculates (approximates) a first area image fᵢ(V+ΔV) when fᵢ(V) is moved by ΔV through Expression (27) and Taylor expansion.

$$\begin{matrix}[ Math . 27 ] & \\ {f}_{i} ( V + Δ V ) = {f}_{i} ( V ) + ∇ {f}_{i}^{T} ( V ) Δ V + ϵ & ( 27 )\end{matrix}$$

In Expression (27), ε of the third term on the right side is a term into which secondary or higher terms in fᵢ(V+ΔV) are summarized and expressed. Similarly to ∇ᵢ(V), fᵢ(V) is a vector with the same number of dimensions as the vector V indicating a change in pixel value located at the same position i in the room coordinate system between the first area image after being minutely moved and the first area image before being moved for each axis in the space covered by the vector V. Similarly to ∇ᵢ(V), when fᵢ(V) is a six-dimensional vector, fᵢ(V) is expressed by Expression (28).

$$\begin{matrix}[ Math . 28 ] & \\ ∇ {f}_{i} ( V ) = \{ \begin{matrix}( {f}_{i} ( V + Δ {V}_{{θ}_{x}} ) - {f}_{i} ( V ) / Δ {θ}_{x} \\ ( {f}_{i} ( V + Δ {V}_{{θ}_{y}} ) - {f}_{i} ( V ) ) / Δ {θ}_{y} \\ ( {f}_{i} ( V + Δ {V}_{{θ}_{z}} ) - {f}_{i} ( V ) ) / Δ {θ}_{z} \\ ( {f}_{i} ( V + Δ {V}_{{t}_{x}} ) - {f}_{i} ( V ) ) / Δ {t}_{x} \\ ( {f}_{i} ( V + Δ {V}_{{t}_{y}} ) - {f}_{i} ( V ) ) / Δ {t}_{y} \\ ( {f}_{i} ( V + Δ {V}_{{t}_{z}} ) - {f}_{i} ( V ) / Δ {t}_{z}\end{matrix} \} \begin{matrix} \\ \\ \\ \\ \\ \end{matrix} & ( 28 )\end{matrix}$$

In Expression (28), Δθx, Δθy, and Δθz denote rotation angles about three axes in the room coordinate system when the three axes are set to the x axis, the y axis, and the z axis, and Δtx, Δty, and Δtz denote amounts of movement along the axes. Elements on the right side of Expression (28) denote pixel values at the positions i in the room coordinate system in the first image. For example, “Expression (29)” which is a first element on the right side of Expression (28) is a pixel value when the pixel fᵢ(V) located at the position i in the room coordinate system in the first image is rotated about the x axis by the rotation angle Δθx. In this example, Expression (30) is expressed by Expression (31). The other elements on the right side of Expression (28) can also be expressed in this way, and detailed description of the elements will be omitted.

$$\begin{matrix}[ Math . 29 ] & \\ {f}_{i} ( V + Δ {V}_{{θ}_{x}} ) & ( 29 )\end{matrix}$$

$$\begin{matrix}[ Math . 30 ] & \\ Δ {V}_{{θ}_{x}} & ( 30 )\end{matrix}$$

$$\begin{matrix}[ Math . 31 ] & \\ Δ {V}_{{θ}_{x}} = {( Δ {θ}_{x} , 0 , 0 , 0 , 0 , 0 )}^{T} & ( 31 )\end{matrix}$$

### (Execution of Registration)

The registrator 110 acquires the first image and the parameters indicating the position and the posture thereof from the first image acquirer 102, the second image and the parameters indicating the position and the posture thereof from the second imaging acquirer 104, and two or more areas from the area acquirer 112 and calculates an amount of misalignment ΔV between the first image and the second image on the basis of the calculation result from the approximate image calculator 115 and the calculation result from the movement cost calculator 117. The registrator 110 outputs a movement amount signal corresponding to the calculated amount of misalignment ΔV. More specifically, the registrator 110 calculates the amount of misalignment ΔV using Expression (32).

$$\begin{matrix}[ Math . 32 ] & \\ Δ V = arg {min}_{Δ V} E ( Δ V , Ω ) & ( 32 )\end{matrix}$$

In Expression (32), Ω is a set including all the positions i of the pixels Iᵢ(V) included in an area in which the first image and the second image overlap in the room coordinate system. The set Ω may be a set of positions indicating a spatial area which is clinically significant when an area of a tumor is irradiated with a treatment beam B such as a PTV, GTV, or an OAR designated by a planner (such as a doctor) of a treatment plan. The set Ω may be a set of positions indicating an area of a space (a sphere, a cubic, or a rectangular parallelepiped) with a predetermined size centered on a beam irradiation position in the room coordinate system. The predetermined size is set on the basis of a size of a patient P or an average size of a human body. The set Ω may be a range obtained by extending a PTV or a GTV with a predetermined scale. In Expression (32), the cost function is defined by Expression (33).

$$\begin{matrix}[ Math . 33 ] & \\ E ( Δ V , Ω ) = {{Σ}_{i ∈ Ω} [ {I}_{i} ( V + Δ V ) - {T}_{i} ( {V}_{plan} ) ]}^{2} + λ {Σ}_{i ∈ Ω} {f}_{i} ( V + Δ V ) & ( 33 )\end{matrix}$$

In Expression (33), Tᵢ(Vₚₗₐₙ) denotes a pixel value in the second image of an arrangement Vₚₗₐₙ at the position i in the room coordinate system. λ is an adjustment parameter. λ is set to, for example, a large value when a risk at the time of treatment is considered to be important. For example, λ may be set to a larger value as the number of pixels included in the set Ω becomes larger. This is because ∇fᵢ(V) of a part with a uniform pixel value in the area is zero and ∇fᵢ(V) in only the vicinity of a boundary is non-zero. Accordingly, the cost is calculated as being relatively low.

The cost function E(ΔV, Ω) used for the registrator 110 to compare the first image and the second image may be a cost function which is set in two spaces which are not connected as expressed by Expression (34).

$$\begin{matrix}[ Math . 34 ] & \\ E ( Δ V , Ω ) = λ E ( Δ V , {Ω}_{1} ) + ( 1 - λ ) E ( Δ V , {Ω}_{2} ) + λ {Σ}_{i ∈ Ω} {f}_{i} ( V + Δ V ) & ( 34 )\end{matrix}$$

The cost function E(ΔV, Ω) used for the registrator 110 to compare the first image and the second image may be a cost function which is expressed by Expression (36) using Function Expression (35) for designating a weight depending on the position i in the room coordinate system.

$$\begin{matrix}[ Math . 35 ] & \\ w ( i ) , i ∈ {ℝ}^{3} & ( 35 )\end{matrix}$$

$$\begin{matrix}[ Math . 36 ] & \\ E ( Δ V , Ω ) = {{Σ}_{i ∈ Ω} [ w ( i ) ( {I}_{i} ( V + Δ V ) - {T}_{i} ( V ) ) ]}^{2} + λ {Σ}_{i ∈ Ω} {f}_{i} ( V + Δ V ) & ( 36 )\end{matrix}$$

In Function Expression (36), w(i) is a function of returning a value based on the position i and a route of the emitted treatment beam B as a return value. For example, the function w(i) is a function of returning a binary value such as “1” when the position i is a position on a route along which the treatment beam B passes and “0” when the position i is not a position on the route along which the treatment beam B passes. The function w(i) may be, for example, a function in which the return value increases as a distance between the position i and the route along which the treatment beam B passes decreases.

The function w(i) may be, for example, a function of returning the position i and a value based on the set of positions indicating a spatial area which is clinically significant when an area of a tumor is irradiated with the treatment beam B such as a PTV, GTV, or an OAR designated by a planner (such as a doctor) of a treatment plan as a return value. The function w(i) may be, for example, a function of returning a binary value such as “1” when the position i is a set of positions indicating a spatial area and “0” when the position i is not a set of positions indicating a spatial area. The function w(i) may be, for example, a function in which the return value increases as the distance between the position i and the spatial area decreases.

Expression (37) is obtained by rewriting Expression (32) using the approximate image acquired by the approximate image calculator 115.

$$\begin{matrix}[ Math . 37 ] & \\ Δ V = arg {min}_{Δ V} [ {{Σ}_{i ∈ Ω} [ {I}_{i} ( V ) + {∇}_{i}^{T} ( V ) Δ V - {T}_{i} ( V ) ]}^{2} + λ {Σ}_{i ∈ Ω} ( {f}_{i} ( V ) + ∇ {f}_{i}^{T} ( V ) Δ V ) ] & ( 37 )\end{matrix}$$

In Expression (37), ε of the third term on the right side of Expression (22) indicating a pixel Iᵢ(V+ΔV) of the approximate image output from the approximate image calculator 115 is ignored. This is because ε into which secondary or higher terms in Expression (22) are summarized and expressed is a very small value and thus does not greatly affect subsequent processes even if it is ignored.

In the right side of Expression (37), when the right side is differentiated with respect to the amount of movement ΔV and is set to 0 in order to calculate a minimum value of the amount of movement ΔV, the amount of movement ΔV is expressed by Expression (38).

$$\begin{matrix}[ Math . 38 ] & \\ Δ V = {H}^{- 1} {Σ}_{i ∈ Ω} [ {∇}_{i} ( V ) ( {T}_{i} ( V ) - {I}_{i} ( V ) - \frac{λ}{2} ∇ {f}_{i}^{T} ( V ) ] & ( 38 )\end{matrix}$$

Here, H on the right side of Expression (38) is a hessian matrix which is defined by Expression (15).

The registrator 110 updates the vector V indicating the position and the posture of the first image with Expression (39) using the amount of movement ΔV calculated using Expression (38).

$$\begin{matrix}[ Math . 39 ] & \\ V ← V + Δ V & ( 39 )\end{matrix}$$

In Expression (39), the updated vector V indicating the position and the posture of the first image is defined as a vector V₁.

The registrator 110 repeats calculation of the amount of movement ΔV using Expression (38) until a change of the updated vector V₁ of the first image becomes small. The time point at which the change of the vector V₁ becomes small is a time point at which a norm of the amount of movement ΔV, that is, the amount of misalignment in position and posture between the first image and the second image, becomes equal to or less than a predetermined threshold value. In other words, a posture of a patient P appearing in the second image is determined to match the posture of the patient P in the treatment planning stage appearing in the first image. The norm of the amount of movement ΔV has only to be a norm of a vector, and, for example, one of a l0 norm, a l1 norm, and a l2 norm is used.

When the set Ω is an area of a PTV or a GTV as described above and the position and the posture of the first image are updated, it is necessary to update elements of the set Ω. That is, this is because the set Ω is a set of coordinate positions in the room coordinate system and thus the position changes with movement of the first image in the room coordinate system. In order to make this update unnecessary, it is preferable that an area defining the set Ω not be included in the first image of which the position and the posture are updated. For example, a CT image (the second image in the aforementioned description) which is captured immediately before treatment may be replaced with a first image, and a CT image (the first image in the aforementioned description) including treatment planning information may be replaced with a second image.

Calculation of the amount of movement ΔV in the registrator 110 may be repeated until the number of times of repeated calculation is greater than a preset number of times. In this case, it is possible to shorten a time required for the registrator 110 to calculate the amount of movement ΔV. However, in this case, at the time point at which the number of times of repeated calculation becomes greater than a preset number of times, the registrator 110 ends calculation of the amount of movement ΔV, but it cannot be said that the norm of the amount of movement ΔV is equal to or less than a predetermined threshold value. In other words, it is also conceivable that there is a high likelihood that calculation of alignment of a patient P will fail. In this case, the registrator 110 may output a warning signal indicating that calculation of the amount of movement ΔV has ended because the number of times of repeated calculation is greater than a preset number of times to, for example, a warning unit (not shown) provided in the medical image processing device 100B or the treatment system 1. Accordingly, the warning unit which is not shown can notify an executor of radiation treatment such as a doctor, that is, a user of the treatment system 1 that there is a likelihood that calculation of alignment of a patient P will fail.

The registrator 110 calculates, the amount of movement ΔV calculated as described above, that is, the amount of misalignment in position and posture between the first image and the second image, for each degree of freedom in Expression (5). Then, the registrator 110 determines an amount of movement (an amount of translation and an amount of rotation) of the bed 12 on the basis of the calculated amount of misalignment for each degree of freedom. At this time, for example, the registrator 110 sums the amount of movement ΔV when an approximate image is calculated from the first image for each degree of freedom. Then, the registrator 110 determines the amount of movement of the bed 12 in which the current position of the patient P is moved by the summed amount of movement for each degree of freedom. Then, the registrator 110 outputs a movement amount signal indicating the determined amount of movement of the bed 12 to the bed controller 14.

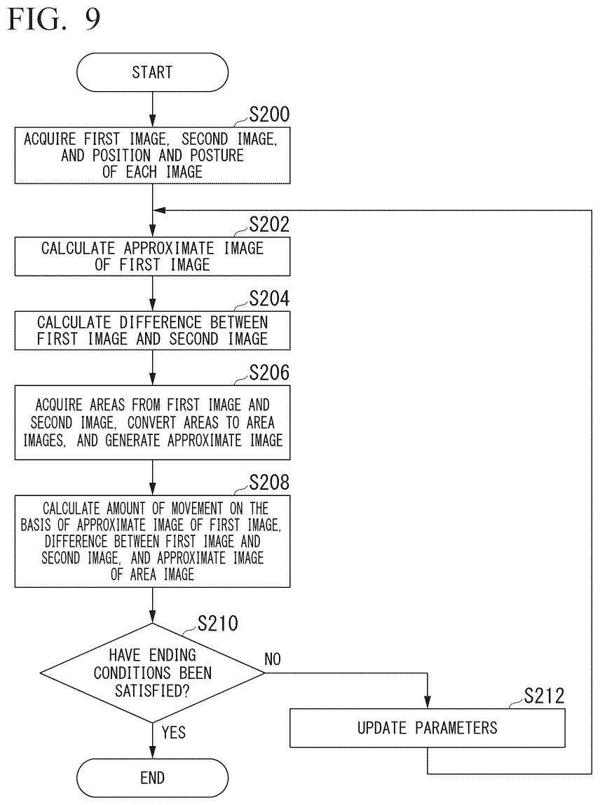

A process flow that is performed by the medical image processing device 100B will be described below with reference to FIG. 9. FIG. 9 is a flowchart showing a process flow that is performed by the medical image processing device 100B. First, the medical image processing device 100B acquires a first image and parameters indicating a position and a posture thereof and a second image and parameters indicating a position and posture thereof using the first image acquirer 102 and the second imaging acquirer 104 (Step S200). Then, the medical image processing device 100B calculates an approximate image of the first image using the approximate image calculator 115 using the aforementioned method (Step S202).

Then, the medical image processing device 100B calculates a difference between the first image and the second image using the image similarity calculator 114 (Step S204). Then, the medical image processing device 100B converts two or more areas corresponding to one or both of the first image and the second image acquired from the area acquirer 112 to an area image including pixel values in which flag information indicating whether each pixel is included in the area is embedded, correlates the area image with the position and the posture of the first image and the second image, and then calculates an approximate image thereof using the movement cost calculator 117 (Step S206).

Then, the medical image processing device 100B calculates an amount of movement ΔV on the basis of the approximate image of the first image, the difference between the first image and the second image, and the approximate image of the area image using the registrator 110 using the aforementioned method (Step S208). Then, the medical image processing device 100B determines whether the calculated amount of movement ΔV satisfies ending conditions (for example, the number of times of calculation is equal to or greater than a predetermined number of times or a difference from the previous value is less than a threshold value as in the flowchart shown in FIG. 6) using the registrator 110 (Step S210). When it is determined that the calculated amount of movement ΔV satisfies the ending conditions, the medical image processing device 100B outputs the confirmed amount of movement ΔV as a movement amount signal. On the other hand, when it is determined that the ending conditions are not satisfied, the medical image processing device 100B updates the parameter x with x=x+Δx using the registrator 110 (Step S212) and returns the process flow to Step S202. Accordingly, the process flow of the flowchart ends.

According to the second embodiment described above, when CT images of a patient captured at the time of treatment planning and at the time of treatment are aligned, a cost is calculated using an approximate image, and a movement amount signal in which the similarity is high and the cost is low is output. Accordingly, it is possible to perform image comparison at a high speed and with high accuracy.

While some embodiments of the present invention have been described above, these embodiments are provided as examples and are not intended to limit the scope of the present invention. These embodiments can be realized in various other forms, and various omissions, substitutions, and modifications can be added thereto without departing from the gist of the present invention. These embodiments and modifications thereof are included in the scope or gist of the present invention and are also included in the inventions described in the appended claims and equivalent scopes thereof.

What is claimed is:

1. A medical image processing device comprising:
a first image acquirer configured to acquire a first image which is captured by imaging an internal body of a patient;
a second image acquirer configured to acquire a second image which is captured by imaging the internal body of the patient at a time point different from that of the first image;
an area acquirer configured to acquire two or more areas corresponding to one or both of the first image and the second image;
an image similarity calculator configured to calculate a similarity between the first image and the second image;
a cost calculator configured to calculate a cost based on a positional relationship between the areas; and
a registrator configured to calculate a relative position of the first image with respect to the second image to increase the similarity and to decrease the cost.

2. The medical image processing device according to claim 1, wherein the registrator comprises:
an approximate image calculator configured to calculate an approximate image which is generated by misaligning the first image by a predetermined width for each degree of freedom in which a posture of the patient changes; and
a movement cost calculator configured to move the area by a predetermined width for each degree of freedom in which the posture of the patient changes and to calculate a change in cost from the area before movement as a movement cost, and
wherein the registrator determines an amount of movement of the first image on a basis of an amount of misalignment between the first image and the second image using the approximate image and an amount of misalignment calculated from the movement cost and outputs a movement amount signal corresponding to the determined amount of movement.

3. The medical image processing device according to claim 1, further comprising an image converter configured to acquire the first image and the second image as integral images by converting an image of the internal body of the patient on a basis of an irradiation route of a treatment beam with which the patient is irradiated.

4. The medical image processing device according to claim 1, wherein the cost is a value based on an area or a volume of a non-overlap part between the two or more areas.

5. The medical image processing device according to claim 4, wherein the cost calculator calculates the cost to increase the cost as the area or the volume of the non-overlap part increases.

6. The medical image processing device according to claim 1, wherein the cost is a shortest distance between the two or more areas.

7. The medical image processing device according to claim 1, wherein the two or more areas comprise a first area corresponding to the first image and a second area corresponding to the second image, and
the area acquirer acquires the second area by modifying the first area.

8. The medical image processing device according to claim 1, wherein the two or more areas comprise an irradiation field of a treatment beam with which the patient is irradiated.

9. The medical image processing device according to claim 1, wherein the two or more areas comprise a tumor in the internal body of the patient.

10. A treatment system comprising:
the medical image processing device according to claim 1; and
a treatment device comprising an irradiator configured to irradiate the patient with radiation, an imaging device configured to capture the first image and the second image, a bed on which the patient is loaded and fixed, and a bed controller configured to control movement of the bed in response to a movement amount signal.

11. A medical image processing method that is performed by a computer, the medical image processing method comprising:
acquiring a first image which is captured by imaging an internal body of a patient;
acquiring a second image which is captured by imaging the internal body of the patient at a time point different from that of the first image;
acquiring two or more areas corresponding to one or both of the first image and the second image;
calculating a similarity between the first image and the second image;
calculating a cost based on a positional relationship between the areas; and
calculating a relative position of the first image with respect to the second image to increase the similarity and to decrease the cost.

12. A computer-readable non-transitory storage medium storing a program for causing a computer to perform:
acquiring a first image which is captured by imaging an internal body of a patient;
acquiring a second image which is captured by imaging the internal body of the patient at a time point different from that of the first image;
acquiring two or more areas corresponding to one or both of the first image and the second image;
calculating a similarity between the first image and the second image;
calculating a cost based on a positional relationship between the areas; and
calculating a relative position of the first image with respect to the second image to increase the similarity and to decrease the cost.
